(19) United States
(12) Patent Application Publication
(10) Pub. No.: US 2019/0059313 A1
(43) Pub. Date: Feb. 28, 2019

(54) Methods for producing a standard form Hydrangea plants and cuttings thereof

(71) Applicants: Ronaldus Wilhelmus VLASVELD, BOSKOOP (NL); Jane Suzanne VLASVELD-PRICE, BOSKOOP (NL); Elias Cornelius Teatse DE BOER, CHILLIWACK (CA); Catharina Maria Jaqueline DE BOER-STOLWIJK, CHILLIWACK (CA)

(72) Inventors: Ronaldus Wilhelmus VLASVELD, BOSKOOP (NL); Jane Suzanne VLASVELD-PRICE, BOSKOOP (NL); Elias Cornelius Teatse DE BOER, CHILLIWACK (CA); Catharina Maria Jaqueline DE BOER-STOLWIJK, CHILLIWACK (CA)

(21) Appl. No.: 16/115,321

(22) Filed: Aug. 28, 2018

(30) Foreign Application Priority Data
Aug. 28, 2017 (NL) 2019455

(51) Int. Cl. A01H 6/48; A01H 5/02; A01H 5/04

(52) U.S. Cl. CPC A01H 6/48; A01H 5/04; A01H 5/02

(57) ABSTRACT

The present invention relates to a method for producing a cutting for a standard form Hydrangea plant. The invention also relates to a method for producing a standard form Hydrangea plant and a flowering standard form Hydrangea plant obtainable with this method. The invention also relates to a Hydrangea cutting for a standard form Hydrangea plant.

The present invention relates to a method for producing a cutting for a standard form Hydrangea plant. The invention also relates to a method for producing a standard form Hydrangea plant and a flowering standard form Hydrangea plant obtainable with this method. The invention also relates to a Hydrangea cutting for a standard form Hydrangea plant.

Hydrangeas are popular ornamental plants, and are well known for their large flower heads. There are approximately 23 species of Hydrangea, of which only a limited number of species are widely cultivated. The most popular species is Hydrangea macrophylla, which is commonly known as bigleaf, French, garden or florist's hydrangea. It produces large inflorescences of white, pink or blue flowers in late spring/early summer.

All hydrangeas normally grow in the form of woody flowering shrubs. However, among florists and gardens a desire exists to grow the flower in tree form. Indeed it is possible to grow hydrangea in the form of a tree, although this requires a number of technical measures. A tree form Hydrangea is commonly referred to in the art as “standard form Hydrangea”. In these forms a flowering crown is grown op top of a single basis stem, which serves as a “standard” for the flowers. Apart from the branches of the crown, this stem typically has no side branches.

A number of ways are known in the art to produce standard form hydrangeas.

A first method is to take a small herbaceous cutting of approximately 10-20 cm from a mother plant during the meteorological summer, for instance in July (northern hemisphere). This small cutting is planted and grown for about a year during which lower side branches are removed so that a plant with an unbranched stem of approximately 50-60 cm long is obtained. In July of the year following planting of the small cutting, the top of the cutting is removed. This results in the plant producing a crown on the top of its stem. Flowering of this crown normally follows in the year after removing the top, suitably in the meteorological spring/summer. With this method it appears only possible to produce stems with a limited length of about 50-60 cm. Further, by using this method it takes a considerable long time of almost 2 years to produce a flowering standard form hydrangea plant from a cutting.

To provide a flowering standard form hydrangea plant within a shorter time frame, a second method has been developed in which essentially wooden stems are used as cuttings. In this method wooden cuttings are taken in the meteorological winter, e.g. February (northern hemisphere). Because of the moment of taking the cuttings the mother plants from which these cuttings are taken are grown in greenhouses to avoid a risk of freezing of buds. As cuttings long straight stems are taken, which are essentially lignified or woodified during this time of season. These cuttings are supported by a stake and grown to allow rooting, which takes a considerable time because at the time of taking the cutting, the stem does not have leaves that can provide energy to the plant to induce and promote rooting. After approximately 1.5 years after planting the cutting, i.e. in the second meteorological spring/summer following taking and planting the cutting, during which period lower side branches and leaves are removed to provide a long straight stem, flowers will grow and a flowering standard form hydrangea plant is obtained.

A third method includes grafting of a crown forming plant part on an existing plant. This way it also takes a number of years to obtain a flowering standard form hydrangea plant. Moreover a horticulturist may be faced with problems relating to incompatibility of the graft and the stem to which it is grafted.

The existing methods for producing flowering standard form hydrangea plants have the main disadvantage that it takes a long time to obtain flowering standard form Hydrangea plants, which is economically disadvantageous for horticulturists that produce and sell these standard form hydrangeas. Furthermore, both the above described first and second methods in general result in standard form hydrangea plants with a maximal stem length of about 60 cm.

The present invention aims to overcome at these advantages.

### DESCRIPTION OF THE INVENTION

In a first aspect therefore the invention relates to method for producing a cutting for a standard form Hydrangea plant, comprising the steps of: pruning a first Hydrangea plant to its base; growing said pruned first Hydrangea plant to produce one or more herbaceous shoots; cutting a said herbaceous shoot to provide an unbranched herbaceous Hydrangea stem with a length of at least 30 cm, wherein said stem comprises a top side comprising a terminal bud and a number of leaves and axillary buds in the axils of said leaves distributed along the stem from the top side to the other side of the stem, and maintaining a number of uppermost leaves and their respective axillary buds on the top side, and removing the remainder of the leaves and axillary buds from said stem.

In a second aspect the invention relates to a method for producing a standard form Hydrangea plant, comprising the steps of: providing a cutting from a first Hydrangea plant; planting said cutting; growing said cutting to allow root formation to provide a second Hydrangea plant; removing the leaves from said second Hydrangea plant which were present at the moment of planting, but maintaining their respective axillary buds; further growing the second Hydrangea plant to produce a standard form Hydrangea plant, wherein said standard Hydrangea plant produces flowers in the year following planting of said cutting; wherein said cutting is an unbranched herbaceous Hydrangea stem with a length of at least 30 cm, wherein said stem comprises a top side comprising a terminal bud and a number of uppermost leaves and their respective axillary buds, the remainder of the originally present leaves and axillary buds having been removed from said stem.

In a third aspect the invention relates to a hydrangea cutting for a standard form Hydrangea plant, which is an unbranched herbaceous Hydrangea stem with a length of at least 30 cm, wherein said stem comprises a top side comprising a terminal bud and a number of uppermost leaves and their respective axillary buds, wherein the remainder of the leaves and axillary buds has been removed from said stem. The hydrangea cutting of the third aspect is obtainable by means of the method of the first aspect.

In a fourth aspect the invention relates to a flowering standard form Hydrangea plant obtainable in accordance with the method of the second aspect of the invention.

For purposes of the invention, preferably Hydrangea macrophylla is used because of its appealing flowers and easy cultivation. Any variety can be used for purposes of this invention.

In order to produce a suitable cutting for purposes of this invention, in a first step a first Hydrangea plant, i.e. mother plant in the context of the present invention, is pruned to its base, for instance up to approximately 20 cm above ground. In practice this first Hydrangea plant is a shrub form Hydrangea plant. This first Hydrangea plant can be suitable grown outside a greenhouse in any amount. It is preferred to perform this pruning step by mowing, for instance by means of a mower or chainsaw. This way, large amounts of Hydrangea plants can be pruned to their basis in a minimal amount of time. The step of pruning said first Hydrangea plant to its basis suitably takes place at the end of meteorological winter or the beginning of meteorological spring. It is preferred that this step of pruning takes place in the last month of the meteorological winter or the first month of the meteorological spring, i.e. February and March, in the northern hemisphere or August in the southern hemisphere, more preferably in the first month of the meteorological spring, i.e. March in the northern hemisphere or August in the southern hemisphere, when no substantial frost is to be expected anymore for the following period.

When reference is made to seasons in this application, it is to be understood that the meteorological meaning is intended. The meteorological winter corresponds to December, January, February in the northern hemisphere, and June, July, August in the southern hemisphere. The meteorological spring corresponds to March, April, May in the northern hemisphere, and September, October, November in the southern hemisphere. The meteorological summer corresponds to June, July, August in the northern hemisphere, and December, January, February in the southern hemisphere.

After the step of pruning the first Hydrangea plant the plants fresh shoots will grow on the plant. In the meteorological summer following the pruning step these shoots are in essentially herbaceous state, are essentially straight and have reached lengths of up to 110 cm and some cases even 150 cm.

At the moment that the shoots have reached a suitable length, herbaceous shoots are cut to provide unbranched herbaceous Hydrangea stems with a length of at least 30 cm. Cutting can be performed by any suitable means, such as for instance pruning shears of knives. The inventors have surprisingly observed that when an unbranched herbaceous Hydrangea stem with a length of at least 30 cm is used as a cutting, a flowering standard form hydrangea plant can be obtained within a year after planting the cutting. Because normally herbaceous shoots with sufficient lengths are reached during the meteorological summer, it is preferred that the step of cutting takes place during the meteorological summer. Further cutting steps may be performed to adapt the stems to the desired size. It is preferred that the cut unbranched herbaceous Hydrangea stem has a length between 30 and 150 cm, more preferably between 30 and 110 cm. Particularly preferable stems have a length between 70 and 100 cm, preferably approximately 80 cm. Suitable stem lengths for instance may be between 30, 35, 40, 45, 50, 55, 60, 65 or 70 to 110 cm, between 30, 35, 40, 45, 50, 55, 60, 65 or 70 to 100 cm, such as between 30, 35, 40, 45, 50, 55, 60, 65 or 70 to 90 cm. For instance between 50 and 80 cm or between 60 and 90 cm, such as for instance about 60, 65, 70, 75, or 80 cm or any length between the above mentioned ranges.

It is preferred that the step of cutting takes place during one of the last two months of the meteorological summer, i.e. July or August in the northern hemisphere or January or February in the southern hemisphere, because then stems have reached optimal lengths of up to 110 cm and in some cases even 150 cm. This way all stems with the preferred lengths as mentioned above can be cut at the same time and lengths can optionally be adapted to one or more desired predetermined lengths. A further advantage of cutting at this stage is that the mother plants from which the cuttings are derived can be grown outside a glass-house, because no freezing of buds occurs at this stage. This saves the costs for a glasshouse. The inventors have further observed that growing the shoots on the mother plant outside a glasshouse results in stems that are thicker, straighter and stiffer than wooden lignified stems grown in a glasshouse. Therefore, when used for growing a standard form Hydrangea plant, the cuttings obtained in accordance with the invention do not require the support of a stake, which saves material and labour.

It is highly preferred that the step of cutting does not take place after the meteorological summer, because then there is a risk that insufficient and/or delayed root formation takes place, which will lead to a delay in flower formation.

The cutting step results an unbranched herbaceous Hydrangea stem. Of this stem a number of uppermost leaves and their respective axillary buds are maintained on the top side, and the remainder of the leaves and axillary buds from said stem is removed, preferably after cutting and before planting. The number of uppermost leaves and their respective axillary buds which are maintained on the cutting depends on the length of the stem and the pot wherein the cutting is planted. The inventors have observed in this respect that the flowers will eventually originate from the remaining buds. Keeping many buds on the top side of the stem will therefore result in a heavy crown and increase the risk of instability of the potted plant in a case of using a pot with a small basis, for instance under windy circumstances or during transport. For this reason it is preferred that between 4 to 10 uppermost leaves and their respective axillary buds on the top side of said stem are maintained on the cutting, while the remainder of the leaves and axillary buds from said stem are removed. It is more preferred that between 6 to 8 uppermost leaves and their respective axillary buds on the top side of said stem are maintained on the cutting, while the remainder of the leaves and axillary buds from said stem are removed. These numbers ensure a compact and appealing flower crown, while maintaining stability.

In accordance with the invention, the above described cuttings can be used for a method for producing a standard form Hydrangea plant according to the second aspect of the invention. It is preferred that the cutting has been obtained in accordance with the steps of the method of the first aspect of the invention. All steps of producing the cutting and growing the plant may be performed at the same plantation. The method of the second aspect of the invention may therefore suitably further comprise the steps of producing a cutting in accordance with the method of the first aspect.

In the method of the second aspect of the invention it is preferred that the cutting is planted in a pot. Suitable sizes, volumes and shapes of pots are commercially available. The pot preferably has a size that is suitable to support growth of a flowering standard form hydrangea plant. This way, growing plants do not have to be transferred between different pots during the process of producing a flowering standard form hydrangea plant.

The cutting is preferably inserted in potting soil to at least 1 cm from the bottom of the pot. This reduces the risk of dehydration. The surface of said potting soil is preferably covered with plastic for a period between 4 to 8 weeks after planting, preferably approximately 6 weeks for the same reason. The inventors have also observed that this promotes root formation.

The step of planting said cutting takes place in the same period as providing the cutting, preferably with a minimum time in between these steps. Accordingly, planting preferably also takes place during the meteorological summer, more preferably during one of the last two months of the meteorological summer, i.e. July or August in the northern hemisphere and January or February in the southern hemisphere, because as mentioned above, at this stage the stems of the shoots grown on the first hydrangea plant or mother plant have reached suitable lengths while still being in essentially herbaceous state. Furthermore, the inventors have observed that planting the cuttings during this period results in better root formation than when cuttings are planted after this period. It is therefore highly preferred that the step of cutting does not take place after the meteorological summer, because then there is a risk that insufficient root formation has taken place after the following winter season, which will lead to a delay in flower formation.

During growth, nutrition or other additives may be added. For instance, aluminium sulphate may be added to induce formation of blue flowers.

During the first meteorological winter following the planting step the plants may hibernate in a glasshouse to prevent freezing of buds and thus delay of flower formation. If no frost occurs, plant may hibernate outside the glasshouse. It is also preferred that during this period, the leaves which were present at the moment of planting are removed, while their respective axillary buds are maintained on the plant. It is preferred that this takes place in the second winter month, i.e. January in the northern hemisphere or July in the southern hemisphere. The inventors have observed that from these buds new branches grow and that branches produce flowers already in the following spring, usually in May and or June (northern hemisphere) of the same year. This way a standard Hydrangea plant is obtained which produces flowers in the year following planting of said cutting.

After removing the leaves in this step fertilization may start to promote growth.

It is noted that it is not required to remove the top of the plant in order to obtain a flowering crown as is common practice in the prior art. The inventors have observed that with the method for producing a standard form hydrangea plant in accordance with the invention, even without this step of removing tops, compact flowering crowns are obtained. Apart from the fact that omitting such a “topping” step saves work, it also results in plants with more compact flower crowns than prior art standard form hydrangeas have.

The invention as described herein provides a way to obtain a flowering standard Hydrangea plant within a period of 10 months or less from planting a cutting. In fact, if a cutting is produced in the northern hemisphere and planted in August of one year, a flowering standard Hydrangea plant can be obtained already in May of the next year, i.e. within a period between 8 and 10 months. Accordingly, the same results will be obtained in the southern hemisphere at the equivalent moments.

The present invention makes it possible to produce a flowering standard form Hydrangea plant in a considerable shorter time than prior art methods. This considerably increases production capacity of standard form Hydrangea plants.

Furthermore, in accordance with the present invention the mother plants from which cuttings are taken will not have to be grown in a glasshouse due to the timing of taking the cutting, i.e. when an herbaceous shoot has a suitable stem length, which is normally in the meteorological summer. Because of this timing, risk of freezing of buds is minimized and flowering standard form Hydrangea plants can be obtained already in the spring following the summer of taking and planting the cutting and immediately be sold to consumers.

Because of the short production time it can be easily determined how long the stem of a cutting should be to obtain a plant with a basis stem or “standard” of the desired length.

The flowers of the flowering standard form Hydrangea plants obtained in accordance with the invention directly result from the uppermost buds that are maintained on the cutting. Because this cutting derives from a young herbaceous shout, these uppermost buds will be in close proximity of each other. During subsequent rooting and hibernation of the plant these distances will not become much larger. As a result, flower crowns of the flowering standard form Hydrangea plants obtained in accordance with the invention are more compact than prior art standard form Hydrangea plants. This is not only more appealing, but it also increases stability of the potted plant and reduces the risk that the plant falls because of strong wind.

1. A method for producing a cutting for a standard form Hydrangea plant, comprising the steps of:
pruning a first Hydrangea plant to its base;
growing said pruned first Hydrangea plant to produce one or more herbaceous shoots;
cutting a said herbaceous shoot to provide an unbranched herbaceous Hydrangea stem with a length of at least 30 cm, wherein said stem comprises a top side comprising a terminal bud and a number of leaves and axillary buds in the axils of said leaves distributed along the stem from the top side to the other side of the stem; and
maintaining a number of uppermost leaves and their respective axillary buds on the top side, and removing the remainder of the leaves and axillary buds from said stem.

2. The method according to claim 1, wherein said unbranched herbaceous Hydrangea stem has a length between 30 and 150 cm.

3. The method according to claim 1, wherein said unbranched herbaceous Hydrangea stem has a length between 30 and 110 cm.

4. The method according to claim 1, wherein said unbranched herbaceous Hydrangea stem has a length between 70 and 100 cm, preferably approximately 80 cm.

5. The method according to claim 1, wherein said step of cutting takes place during the meteorological summer.

6. The method according to claim 1, wherein said step of pruning said first Hydrangea plant to its base takes place during the last month of the meteorological winter or the first month of the meteorological spring preceding said step of cutting said herbaceous shoot.

7. The method according to claim 1, comprising maintaining between 4 to 10 uppermost leaves and their respective axillary buds on the top side of said stem, and removing the remainder of the leaves and axillary buds from said stem.

8. The method according claim 7, comprising maintaining between 6 to 8 uppermost leaves and their respective axillary buds on the top side, and removing the remainder of the leaves and axillary buds from said stem.

9. The method according to claim 1, wherein said first Hydrangea plant is a shrub form Hydrangea plant.

10. The method according to claim 1, wherein said step of cutting said herbaceous shoot takes place during one of the last two months of the meteorological summer.

11. A method for producing a standard form Hydrangea plant, comprising the steps of:
providing a cutting from a first Hydrangea plant;
planting said cutting;
growing said cutting to allow root formation to provide a second Hydrangea plant;
removing the leaves from said second Hydrangea plant which were present at the moment of planting, but maintaining their respective axillary buds;
further growing the second Hydrangea plant to produce a standard Hydrangea plant, wherein said standard Hydrangea plant produces flowers in the year following planting of said cutting;
wherein said cutting is an unbranched herbaceous Hydrangea stem with a length of at least 30 cm, wherein said stem comprises a top side comprising a terminal bud and a number of uppermost leaves and their respective axillary buds, the remainder of the originally present leaves and axillary buds having been removed from said stem.

12. The method according to claim 11, wherein the step of removing the leaves from said second Hydrangea plant takes place during the first meteorological winter following said planting step.

13. The method according to claim 11, wherein the step of planting said cutting takes place during the meteorological summer.

14. The method according to claim 11, wherein said cutting has been produced by the steps of:
pruning a first Hydrangea plant to its base;
growing said pruned first Hydrangea plant to produce one or more herbaceous shoots;
cutting a said herbaceous shoot to provide an unbranched herbaceous Hydrangea stem with a length of at least 30 cm, wherein said stem comprises a top side comprising a terminal bud and a number of leaves and axillary buds in the axils of said leaves distributed along the stem from the top side to the other side of the stem; and
maintaining a number of uppermost leaves and their respective axillary buds on the top side, and removing the remainder of the leaves and axillary buds from said stem.

15. The method according to claim 14, wherein the method comprises further steps of producing a cutting by the steps of:
pruning a first Hydrangea plant to its base;
growing said pruned first Hydrangea plant to produce one or more herbaceous shoots;
cutting a said herbaceous shoot to provide an unbranched herbaceous Hydrangea stem with a length of at least 30 cm, wherein said stem comprises a top side comprising a terminal bud and a number of leaves and axillary buds in the axils of said leaves distributed along the stem from the top side to the other side of the stem; and
maintaining a number of uppermost leaves and their respective axillary buds on the top side, and removing the remainder of the leaves and axillary buds from said stem.

16. The method according to claim 11, wherein said cutting is planted and grown in a pot, and wherein said cutting is inserted in potting soil to at least 1 cm from the bottom of the pot.

17. The method according to claim 16, wherein the surface of said potting soil is covered with plastic for a period between 4 to 8 weeks, preferably approximately 6 weeks.

18. The method according to claim 11, wherein a flowering standard Hydrangea plant is obtained within a period of 10 months or less from planting the cutting, preferably within a period between 8 and 10 months.

19. The method according to claim 1, wherein said Hydrangea plant is a Hydrangea macrophylla.

20. A Hydrangea cutting for a standard form Hydrangea plant, which is an unbranched herbaceous Hydrangea stem with a length of at least 30 cm, wherein said stem comprises a top side comprising a terminal bud and a number of uppermost leaves and their respective axillary buds, wherein the remainder of the leaves and axillary buds has been removed from said stem.

21. The Hydrangea cutting according to claim 20, wherein said unbranched herbaceous Hydrangea stem has a length between 30 and 150 cm.

22. The Hydrangea cutting according to claim 20, wherein said unbranched herbaceous Hydrangea stem has a length between 30 and 110 cm.

23. The Hydrangea cutting according to claim 20, wherein said unbranched herbaceous Hydrangea stem has a length between 70 and 100 cm, preferably approximately 80 cm.

24. The Hydrangea cutting according to claim 20, which is derived from a Hydrangea macrophylla plant.

25. The Hydrangea cutting according to claim 20, which has been obtained by the steps of:
pruning a first Hydrangea plant to its base;
growing said pruned first Hydrangea plant to produce one or more herbaceous shoots;
cutting a said herbaceous shoot to provide an unbranched herbaceous Hydrangea stem with a length of at least 30 cm, wherein said stem comprises a top side comprising a terminal bud and a number of leaves and axillary buds in the axils of said leaves distributed along the stem from the top side to the other side of the stem; and
maintaining a number of uppermost leaves and their respective axillary buds on the top side, and removing the remainder of the leaves and axillary buds from said stem.

26. A flowering standard form Hydrangea plant obtainable by the steps of:
providing a cutting from a first Hydrangea plant;
planting said cutting;
growing said cutting to allow root formation to provide a second Hydrangea plant;
removing the leaves from said second Hydrangea plant which were present at the moment of planting, but maintaining their respective axillary buds;
further growing the second Hydrangea plant to produce a standard Hydrangea plant, wherein said standard Hydrangea plant produces flowers in the year following planting of said cutting;
wherein said cutting is an unbranched herbaceous Hydrangea stem with a length of at least 30 cm, wherein said stem comprises a top side comprising a terminal bud and a number of uppermost leaves and their respective axillary buds, the remainder of the originally present leaves and axillary buds having been removed from said stem.

27. The method according to claim 12, wherein the step of planting said cutting takes place during the meteorological summer.

28. The method according to claim 11, wherein said Hydrangea plant is a Hydrangea macrophylla.
